(19) United States
(12) Patent Application Publication
(10) Pub. No.: US 2024/0216608 A1
(43) Pub. Date: Jul. 4, 2024

(54) Smart Monitoring of an Intravenous Access Site Using Sensors for Continuous Monitoring

(71) Applicant: Becton, Dickinson And Company, Franklin Lakes, NJ (US)

(72) Inventors: Shishir Prasad, Ramsey, NJ (US); Andrea Bowdon, Baltimore, MD (US); Edward R. Prescott, Baltimore, MD (US); Mariano Mumpower, Sparks, MD (US); Jeffrey Manfred Gunnarsson, Baltimore, MD (US)

(21) Appl. No.: 18/684,130

(22) PCT Filed: Aug. 19, 2022
(86) PCT No.: PCT/US22/40909

§ 371 Date: Feb. 15, 2024

Related U.S. Application Data
(60) Provisional application No. 63/234,976, filed on Aug. 19, 2021.

(51) Int. Cl. A61M 5/168; A61M 5/142

(52) U.S. Cl. CPC A61M 5/16836; A61M 5/142; A61M 2205/0227; A61M 2205/0233; A61M 2205/15; A61M 2205/18; A61M 2205/3303; A61M 2205/3306; A61M 2205/3317; A61M 2205/3327; A61M 2205/3368; A61M 2205/3375; A61M 2205/35; A61M 2205/50; A61M 2205/8206; A61M 2230/005

(57) ABSTRACT

A system for monitoring an intravenous (IV) access site for catheter insertion into a patient's skin. The system may include one or more of a dressing layer configured to cover at least a portion of the IV access site, a cuff configured to be disposed at least partially around the dressing layer, and a camera positioned external to the IV access site. At least one of the dressing layer and the cuff may include at least one embedded component configured for use in detecting one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer.

### CROSS-REFERENCE TO RELATED APPLICATION

The present application claims priority to U.S. Provisional Application Ser. No. 63/234,976, filed Aug. 19, 2021, entitled “Smart Monitoring of an Intravenous Access Site Using Sensors for Continuous Monitoring”, the entire disclosure of which is hereby incorporated by reference in its' entirety.

### BACKGROUND OF THE INVENTION

### Field of the Invention

The present disclosure relates to a system and method of smart monitoring of an intravenous (IV) access site. More specifically, the present disclosure relates to a smart monitoring system capable of continuous monitoring of the IV access site. The system may be capable of alerting a patient and/or care provider of complications related to the IV access site and/or shutting down an infusion pump based upon feedback received from the smart monitoring system.

### Description of Related Art

IV access sites (i.e., catheter insertion sites) require regular site assessments in order to ensure proper placement and patient safety related to the catheter insertion. Typically, these site assessments involve routine visual observations performed by nurses, physicians, technicians, and/or other care providers on patients having an intravenous catheter in place. The nurse or other care provider may inspect the IV access site to confirm, e.g., that the catheter is properly functioning, the catheter is properly in place and has not been dislodged, the dressing is secure and is not peeling, dirty, or otherwise compromised, and/or the skin around the IV access site is not discolored, warm, or soft, indicating a possible infection at the site.

Dressings are generally placed over the IV access site in order to help prevent any catheter-related complications such as, e.g., infiltrations, extravasations, phlebitis, dislodgement, etc. The dressings also help to prevent local site infections by keeping the site clean and dry. In addition to covering the entry site on the patient's skin, dressings may also serve to help maintain catheter placement by securing the catheter to the skin.

While routine observation of the IV access site by care providers may be sufficient in some cases, it still necessitates time and know-how, and the period between such observations can include catheter-related complications which may go undiscovered until next site inspection by the care provider. Additionally and/or alternatively, the observation of the IV access site may necessitate removal of the dressing, thereby increasing the likelihood of site contamination.

### SUMMARY OF THE INVENTION

Accordingly, the present disclosure relates to a smart monitoring system capable of continuous monitoring of the IV access site. The system may be capable of alerting a patient and/or care provider of complications related to the IV access site and/or shutting down an infusion pump based upon feedback received from the smart monitoring system. Sensors utilized in the smart monitoring system may be employed directly on or within the dressing or embedded in, e.g., a cuff configured to be worn over the dressing. Additionally and/or alternatively, in accordance with another aspect of the disclosure, the smart monitoring system may monitor the IV access site via, e.g., one or more cameras placed away from the IV access site itself.

In accordance with an aspect of the present disclosure, a system for monitoring an intravenous (IV) access site for catheter insertion into a patient's skin is disclosed, the system includes a dressing layer configured to cover at least a portion of the IV access site, wherein the dressing layer includes at least one embedded component configured for use in detecting one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer.

In some embodiments, the at least one embedded component includes a plurality of conductive traces.

In some embodiments, the at least one embedded component includes a capacitive film layer.

In some embodiments, the at least one embedded component includes at least two piezoelectric film transducers.

In some embodiments, the at least one embedded component includes a plurality of conductive or piezoresistive ink traces.

In some embodiments, the at least one embedded component includes a first layer having transparent electrodes extending in a first direction and a second layer having transparent electrodes extending in a second direction perpendicular to the first direction.

In some embodiments, the at least one embedded component includes a plurality of piezoresistive film layers.

In some embodiments, the at least one embedded component includes a photoelastic film.

In some embodiments, the at least one embedded component includes an array of printed photodetectors.

In some embodiments, the at least one embedded component includes at least two electrodes configured to be positioned on opposite sides of the catheter.

In some embodiments, the dressing layer further includes an integrated power source and communication electronics.

In some embodiments, based upon the detection of one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer, the system is further configured to initiate at least one of the shutdown of an infusion pump fluidly coupled the catheter and send at least one of an audible or visual alert to a care provider.

In accordance with another aspect of the present disclosure, another system for monitoring an intravenous (IV) access site for catheter insertion into a patient's skin is disclosed. The system includes a dressing layer configured to cover at least a portion of the IV access site, and a cuff configured to be disposed at least partially around the dressing layer. The cuff includes at least one embedded component configured for use in detecting one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer.

In some embodiments, the at least one embedded component includes a plurality of optical sensors.

In some embodiments, the at least one embedded component includes a pair of ultrasonic transducers.

In some embodiments, the at least one embedded component includes at least two photodetectors.

In some embodiments, the cuff further includes a wireless communication device, a microcontroller, and a power source.

In some embodiments, based upon the detection of one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer, the system is further configured to initiate at least one of the shutdown of an infusion pump fluidly coupled the catheter and send at least one of an audible or visual alert to a care provider.

In accordance with another aspect of the present disclosure, another system for monitoring an intravenous (IV) access site for catheter insertion into a patient's skin is disclosed. The system includes a dressing layer configured to cover at least a portion of the IV access site, and a camera positioned externally from the IV access site and configured to capture images of the IV access site, wherein the camera is configured for use in detecting one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer.

In some embodiments, the camera is a high-resolution photographic camera.

In some embodiments, the camera is an infrared camera.

In some embodiments, based upon the detection of one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer, the system is further configured to initiate at least one of the shutdown of an infusion pump fluidly coupled the catheter and send at least one of an audible or visual alert to a care provider.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1A is a top plan view of a dressing layer having conductive traces embedded therein in accordance with an aspect of the present disclosure;

FIG. 1B is a top plan view of an IV access site utilizing the dressing layer of FIG. 1A;

FIG. 2A is a top plan view of a dressing layer having conductive traces and an electrolyte film pad embedded therein in accordance with another aspect of the present disclosure;

FIG. 2B is a top plan view of an IV access site utilizing the dressing layer of FIG. 2A;

FIG. 3A is a top plan view of a dressing layer having a conductive trace layer embedded therein in accordance with another aspect of the present disclosure;

FIG. 3B is a top plan view of an IV access site utilizing the dressing layer of FIG. 3A;

FIG. 4A is a side cross-sectional view of a cuff and dressing layer having optical transmitters and detectors embedded therein in accordance with another aspect of the present disclosure;

FIG. 4B is a top plan view of an IV access site utilizing the cuff and dressing layer of FIG. 4A;

FIG. 5A is a side cross-sectional view of a dressing layer having piezoelectric (PVDF) film transducers embedded therein in accordance with another aspect of the present disclosure;

FIG. 5B is a top plan view of an IV access site utilizing the dressing layer of FIG. 5A;

FIG. 6A is a side cross-sectional view of a cuff and dressing layer having ultrasonic transducers embedded therein in accordance with another aspect of the present disclosure;

FIG. 6B is a top plan view of an IV access site utilizing the cuff and dressing layer of FIG. 6A;

FIG. 7A is a top plan view of a dressing layer having conductive or piezoresistive ink traces printed thereon in accordance with another aspect of the present disclosure;

FIG. 7B is a top plan view of an IV access site utilizing the dressing layer of FIG. 7A;

FIG. 8A is an isometric view of a dressing layer acting as a capacitive touch sensor in accordance with another aspect of the present disclosure;

FIG. 8B is a top plan view of an IV access site utilizing the dressing layer of FIG. 8A;

FIG. 9A is an isometric view of a dressing layer having piezoresistive film layers in accordance with another aspect of the present disclosure;

FIG. 9B is a top plan view of an IV access site utilizing the dressing layer of FIG. 9A;

FIG. 10A is a top plan view of a dressing layer having a photoelastic film in accordance with another aspect of the present disclosure;

FIG. 10B is a top plan view of an IV access site utilizing the dressing layer of FIG. 10A;

FIG. 11A is a top plan view of a dressing layer having printed marker dots on a surface thereof in accordance with another aspect of the present disclosure;

FIG. 11B is a top plan view of a camera and an IV access site utilizing the dressing layer of FIG. 11A;

FIG. 12A is a top plan view of a dressing layer having a printed thermocouple embedded therein in accordance with another aspect of the present disclosure;

FIG. 12B is a top plan view of an IV access site utilizing the dressing layer of FIG. 12A;

FIG. 13A is an example of the use of infrared thermography to measure a temperature-sensitive image of a wound site in accordance with an aspect of the present disclosure;

FIG. 13B is a top plan view of a camera and an IV access site utilizing infrared temperature-sensitive technology in accordance with an aspect of the present disclosure;

FIG. 14A is an example of a portion of a dressing layer having printed photodetectors embedded therein in accordance with an aspect of the present disclosure;

FIG. 14B is a top plan view of an IV access site utilizing the dressing layer of FIG. 14A;

FIG. 15A is a cross-sectional view of a color sensing filter layer and sensor array embedded in a dressing layer in accordance with an aspect of the present disclosure;

FIG. 15B is a top plan view of a camera and an IV access site utilizing the dressing layer of FIG. 15A;

FIG. 16A is a top plan view of a dressing layer having a conductive trace perimeter embedded therein in accordance with another aspect of the present disclosure;

FIG. 16B is a top plan view of an IV access site utilizing the dressing layer of FIG. 16A;

FIG. 17A is side cross-sectional view of a cuff and dressing layer having optical sensors embedded therein in accordance with another aspect of the present disclosure;

FIG. 17B is a top plan view of an IV access site utilizing the cuff and dressing layer of FIG. 17A;

FIG. 18A is a pair of images representing catheter movement in accordance with an aspect of the present disclosure;

FIG. 18B is a top plan view of a camera and IV access site utilizing machine vision to determine catheter movement in accordance with an aspect of the present disclosure; and

FIG. 19 is a top plan view of an IV access site having a dressing layer with a pair of electrodes embedded therein to determine catheter movement in accordance with another aspect of the present disclosure.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

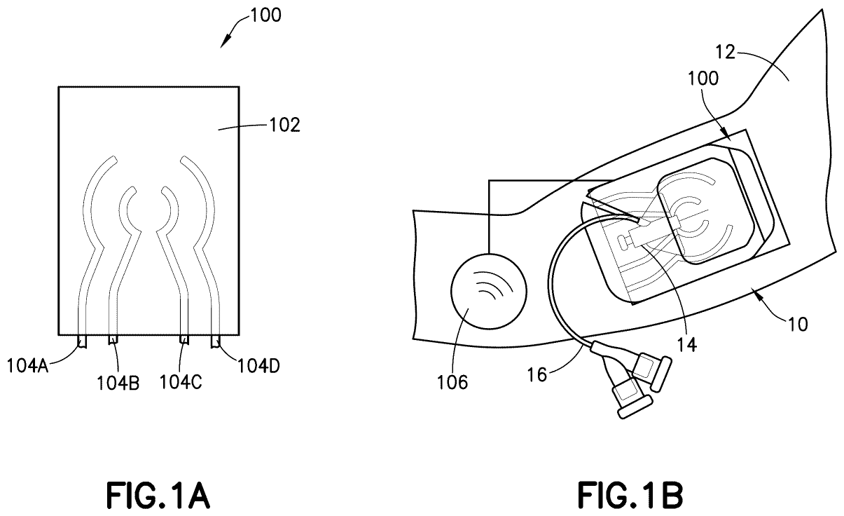

As will be described in detail below with respect to FIGS. 1A-19, the present disclosure relates to various systems configured for continuous monitoring of the IV access site to identify signs of various possible complications. The system(s) may form a closed loop with the IV infusing equipment and/or care providers, switching off the infusing pumps and/or notifying the care providers if such complications are detected. Examples of possible complications capable of being monitored by the systems disclosed herein are, e.g., swelling of the IV access site, temperature changes at the IV access site, infiltration/extravasation from the catheter, fluid detection on top of the patient's skin at the skin piercing location, catheter dislodgement, skin blanching or redness at the IV access site, presence of microbes at the IV access site, and edge lift of the dressing resulting in the exposure of IV site to the external environment. However, it is to be understood that the systems disclosed herein are not limited by these examples.

In some aspects of the present disclosure, sensor(s) for the detection of signs of IV access complication are employed directly on (or embedded in) the dressing layer covering the IV access site.

Additionally and/or alternatively, the sensor(s) may be positioned on (or embedded in), e.g., a flexible cuff that can be worn over of the dressing layer. Unlike the incorporation of such sensor(s) into a dressing layer, which is limited to use with a single patient, incorporation of the sensor(s) in a cuff may enable use with multiple patients, as the cuffs may be sterilized and/or recharged.

In other aspects of the present disclosure, sensing methods positioned away from the IV site, and even away from the patient's body, may be employed. For example, an external camera positioned above or beside the patient may be configured to capture images of the IV access site at regular intervals, utilizing various image processing capabilities to detect possible signs of IV access complications.

The sensor(s) may be powered by any appropriate method, such as an integrated battery or a wired connection. If a wired connection is utilized, the same wire may also be used for data communication. If a battery is used, battery may either be rechargeable or single use. Furthermore, in case of a battery-powered sensors, the electronic box holding the battery (or batteries) may also be configured to hold the appropriate communication circuitry to transfer sensor data wirelessly to a telemetry notification system for data processing and notification for potential complication signature.

Additionally, while not shown in all embodiments described herein for ease of illustration, it is to be understood that all sensors embedded in/on a dressing layer in any of the embodiments described herein may be coupled via thin communication wire to a small electronic patch containing, e.g., a power source, communication electronics, etc. One or both of the electronic patch or the care provider's notification system would further include software to receive and process the provided data. This software may be capable of filtering noise and false alarms when a signal goes above a particular threshold limit.

Referring to FIGS. 1A and 1B, a dressing layer 100 in accordance with an aspect of the present disclosure is shown. The dressing layer 100 is configured to substantially cover an IV access site 10, which is the site on the patient's skin 12 in which an IV catheter 14 is inserted. As described above, the dressing layer 100 is not only configured to protect the IV access site 10, but may also aid in retention and/or positioning of the IV catheter 14 and/or a tube set 16 fluidly coupled to the IV catheter 14.

Furthermore, as shown in FIG. 1A, the dressing layer 100 includes a flexible, primary layer 102 and a plurality of conductive traces 104A, 104B, 104C, 104D. The conductive traces 104A, 104B, 104C, 104D may be embedded in a skin-contact side of the primary layer 102. Furthermore, while not shown, it is to be understood that the conductive traces 104A, 104B, 104C, 104D (and the dressing layer 100) are coupled to a power source such as, e.g., a battery or wired connection.

In one aspect of the present disclosure, the conductive traces 104A, 104B, 104C, 104D may be utilized to detect moisture (i.e., liquid effluent on the patient's skin) surrounding the IV access site 10. Each conductive trace 104A, 104B, 104C, 104D is capable of undergoing a resistance change when in contact with moisture, and with multiple conductive traces 104A, 104B, 104C, 104D, comparative readings across the dressing layer 100 may be made. In this way, when one or more conductive traces 104A, 104B, 104C, 104D detect liquid effluent on the patient's skin at the IV access site 10, the system is configured to communicate with one or both of the infusing equipment and the care provider via, e.g., a communication module 106. The system may then be configured to shut down, e.g., the infusion pump coupled to the tube set 16, send an audible and/or visual alert to the care provider, etc., when it is determined that moisture is present below the dressing layer 100, signifying a possible complication at the IV access site 10. Accordingly, the dressing layer 100 is configured to continuously monitor the IV access site 10 for moisture, thereby avoiding the need for the care provider to routinely check the IV access site 10 via visual inspection, remove the dressing layer 100, etc.

Furthermore, in addition to moisture detection, the conductive traces 104A, 104B, 104C, 104D may be utilized to detect changes in temperature surrounding the IV access site 10, which may signify complications such as infection at the IV access site 10. Each conductive trace 104A, 104B, 104C, 104D is capable of undergoing a resistance change due to temperature changes, and with multiple conductive traces 104A, 104B, 104C, 104D, comparative readings across the dressing layer 100 may be made. In this way, when one or more conductive traces 104A, 104B, 104C, 104D detect temperature changes at the IV access site 10, the system is configured to communicate with one or both of the infusing equipment and the care provider via, e.g., a communication module 106. The system may then be configured to shut down, e.g., the infusion pump coupled to the tube set 16, send an audible and/or visual alert to the care provider, etc., when it is determined that there is a predetermined change in temperature below the dressing layer 100, signifying a possible complication (e.g., an infection) at the IV access site 10.

Alternatively, in some embodiments, conductive traces 104A, 104B, 104C, 104D may be configured as thermally conductive members embedded into the primary layer 102 and utilized only to detect changes in temperature surrounding the IV access site 10. In this way, the temperature measurement can be made accurately within the dressing layer 100 itself. Alternatively, separate temperature responsive traces may be applied to measure temperature changes. Temperature changes can be measured at multiple locations to differentiate local temperature change from environmental temperature changes.

In yet another aspect of the present disclosure, the conductive traces 104A, 104B, 104C, 104D may be configured to measure bio-impedance in order to detect possible fluid build-up (i.e., infiltration) below the patient's skin at the IV access site 10. In such a configuration, the conductive traces 104A, 104B, 104C, 104D may be configured to act as electrodes. If and when it is determined that there is infiltration at the IV access site 10, the system may then be configured to shut down, e.g., the infusion pump coupled to the tube set 16, send an audible and/or visual alert to the care provider, etc. Alternatively, separate traces to measure bio-impedance changes may be applied to measure such changes due to fluid build-up below the patient's skin.

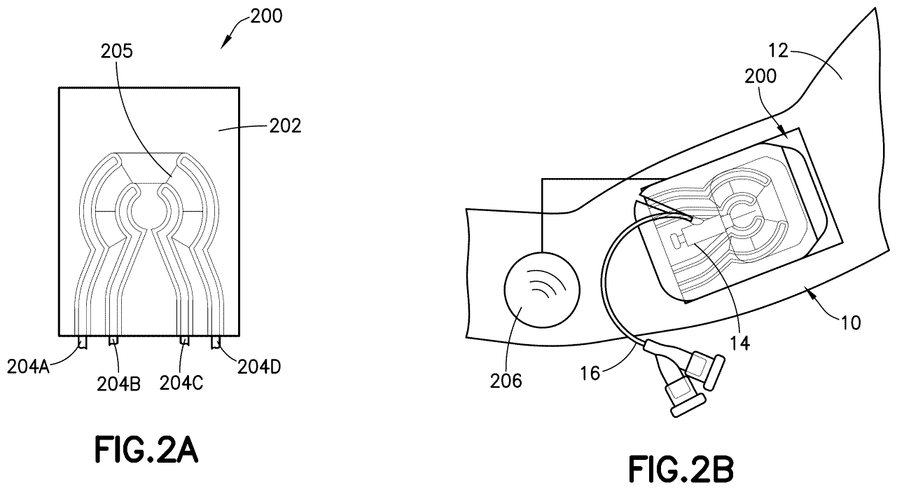

Next, referring to FIGS. 2A and 2B, a dressing layer 200 in accordance with another aspect of the present disclosure is shown. Similar to dressing layer 100 described above, dressing layer 200 is configured to substantially cover an IV access site 10, which is the site on the patient's skin 12 in which an IV catheter 14 is inserted.

Furthermore, as shown in FIG. 2A, the dressing layer 200 includes a flexible, primary layer 202 and an electrolyte film layer 205. The electrolyte film layer 205 comprises a plurality of conductive traces 204A, 204B, 204C, 204D, with materially between these traces creating a new conductive path when in contact with and/or saturated with liquid. The electrolyte film layer 205 and conductive traces 204A, 204B, 204C, 204D may be embedded in a skin-contact side of the primary layer 202. Furthermore, while not shown, it is to be understood that the conductive traces 204A, 204B, 204C, 204D (and the dressing layer 200) are coupled to a power source such as, e.g., a battery or wired connection.

In one aspect of the present disclosure, the conductive traces 204A, 204B, 204C, 204D may be utilized to detect moisture (i.e., liquid effluent on the patient's skin) absorbed by the electrolyte film layer 205 and surrounding the IV access site 10. Each conductive trace 204A, 204B, 204C, 204D is capable of undergoing a resistance change when in contact with moisture, and with multiple conductive traces 204A, 204B, 204C, 204D, comparative readings across the dressing layer 200 may be made. In this way, when one or more conductive traces 204A, 204B, 204C, 204D detect liquid effluent on the patient's skin at the IV access site 10, the system is configured to communicate with one or both of the infusing equipment and the care provider via, e.g., a communication module 206. The system may then be configured to shut down, e.g., the infusion pump coupled to the tube set 16, send an audible and/or visual alert to the care provider, etc., when it is determined that moisture is present below the dressing layer 200, signifying a possible complication at the IV access site 10. Accordingly, the dressing layer 200 is configured to continuously monitor the IV access site 10 for moisture, thereby avoiding the need for the care provider to routinely check the IV access site 10 via visual inspection, remove the dressing layer 200, etc.

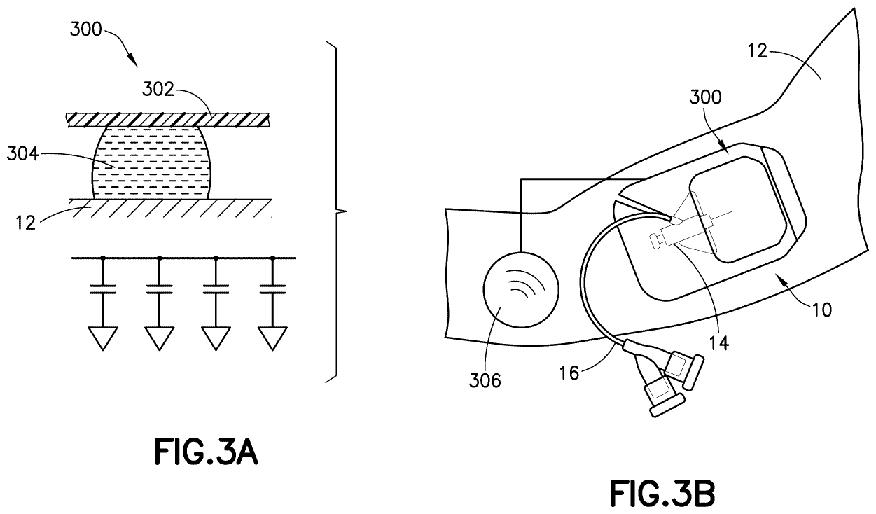

Referring now to FIGS. 3A and 3B, a dressing layer 300 in accordance with another aspect of the present disclosure is shown. As with the other embodiments described above, dressing layer 300 is configured to substantially cover the IV access site 10, which is the site on the patient's skin 12 in which the IV catheter 14 is inserted.

Unlike dressing layers 100 and 200 described above, which utilized conductive traces embedded therein, dressing layer 300 utilizes a capacitive film layer 302 on a skin-contacting side of the dressing layer. The capacitive film layer 302 is flexible and is configured to substantially cover the IV access site 10. Furthermore, while not shown, it is to be understood that the capacitive film layer 302 is coupled to a power source such as, e.g., a battery or wired connection.

In one aspect of the present disclosure, the capacitive film layer 302 may be utilized to detect moisture (i.e., liquid effluent on the patient's skin) surrounding the IV access site 10. That is, capacitance between the capacitive film layer 302 and the patient's skin 12 changes in the presence of a fluid 304 (i.e., liquid effluent). In this way, when the capacitive film layer 302 detects liquid effluent on the patient's skin at the IV access site 10, the system is configured to communicate with one or both of the infusing equipment and the care provider via, e.g., a communication module 306. The system may then be configured to shut down, e.g., the infusion pump coupled to the tube set 16, send an audible and/or visual alert to the care provider, etc., when it is determined that moisture is present below the capacitive film layer 302, signifying a possible complication at the IV access site 10. Accordingly, the dressing layer 300 is configured to continuously monitor the IV access site 10 for moisture, thereby avoiding the need for the care provider to routinely check the IV access site 10 via visual inspection, remove the dressing layer 300, etc. Furthermore, while not shown, it is to be understood that the capacitive film layer 302 may be utilized in conjunction with one or more conductive traces such as those described above with respect to FIGS. 1A-2B.

Furthermore, in addition to moisture detection, the capacitive film layer 302 may also be utilized to detect changes in temperature surrounding the IV access site 10, as the capacitance changes due to temperature changes. In this way, when the capacitive film layer 302 detects temperature changes at the IV access site 10, the system is configured to communicate with one or both of the infusing equipment and the care provider via, e.g., a communication module 306. The system may then be configured to shut down, e.g., the infusion pump coupled to the tube set 16, send an audible and/or visual alert to the care provider, etc., when it is determined that there is a predetermined change in temperature below the dressing layer 300, signifying a possible complication (e.g., an infection) at the IV access site 10. It is to be understood that multiple sensing methods, and at locations different from the IV access site 10, may be applied to separately ascertain the impact of temperature change, swelling, and moisture.

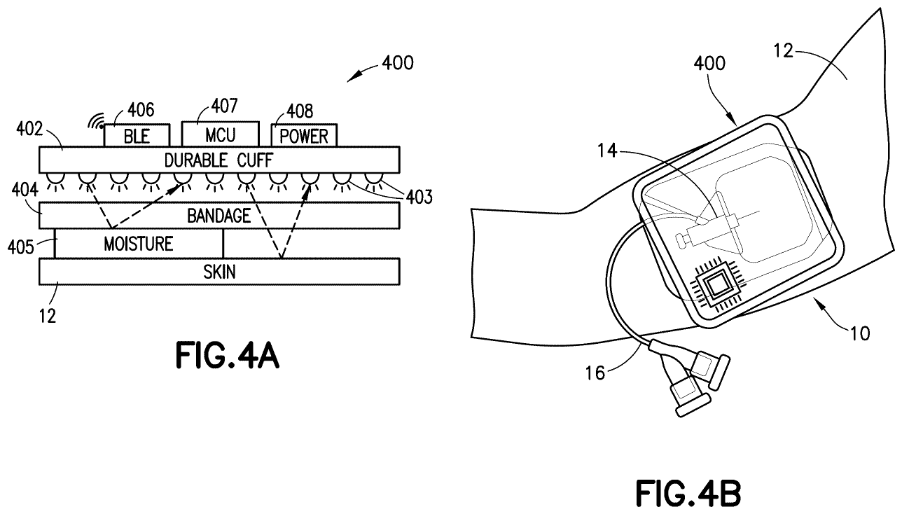

Next, referring to FIGS. 4A and 4B, a cuff and dressing layer assembly 400 in accordance with another aspect of the present disclosure is illustrated. While the dressing layers described above with respect to FIGS. 1A-3B included embedded or otherwise incorporated means of moisture detection, cuff and dressing layer assembly 400 utilizes a separate cuff 402 having a plurality of optical sensors 403 embedded therein. Accordingly, the dressing layer 404 itself does not need to incorporate sensors, conductive traces, etc., and may be configured as a substantially conventional dressing layer.

As with the other embodiments described above, the cuff and dressing layer assembly 400 is configured to substantially cover the IV access site 10, which is the site on the patient's skin 12 in which the IV catheter 14 is inserted. In some embodiments, the cuff 402 may be configured to extend at least partially around, e.g., the patient's arm in order to remain in place. In addition to embedded optical sensors 403, the cuff 402 may include various electronics incorporated therein or thereon such as, e.g., a wireless communication device 406 (e.g., a Bluetooth® low energy (BLE) device), a microcontroller 407, a power source 408 (e.g., a battery).

In one aspect of the present disclosure, the cuff and dressing layer assembly 400 may be utilized to detect moisture (i.e., liquid effluent on the patient's skin) surrounding the IV access site 10. That is, the optical sensors 403 may be configured to both emit and detect light, with the light patterns across the dressing layer 404 and the patient's skin 12 changing in the presence of a fluid 405 (i.e., liquid effluent). In this way, when cuff 402 detects liquid effluent on the patient's skin at the IV access site 10, the system is configured to communicate with one or both of the infusing equipment and the care provider via, e.g., the wireless communication device 406. The system may then be configured to shut down, e.g., the infusion pump coupled to the tube set 16, send an audible and/or visual alert to the care provider, etc., when it is determined that moisture is present below the dressing layer 404, signifying a possible complication at the IV access site 10. Accordingly, the cuff 402 is configured to continuously monitor the IV access site 10 for moisture, thereby avoiding the need for the care provider to routinely check the IV access site 10 via visual inspection, remove the dressing layer 404, etc.

In an alternative embodiment, the optical sensors 403 may instead be configured as infrared (IR) temperature sensors capable of monitoring temperature changes at or near the IV access site 10. Thus, if/when the sensors 403 detect temperature changes at the IV access site 10, the system may be configured to communicate with one or both of the infusing equipment and the care provider via, e.g., a communication module 406. The system may then be configured to shut down, e.g., the infusion pump coupled to the tube set 16, send an audible and/or visual alert to the care provider, etc., when it is determined that there is a predetermined change in temperature below the dressing layer 400, signifying a possible complication (e.g., an infection) at the IV access site 10.

In yet another aspect of the present disclosure, the optical sensors 403 may configured to sense and monitor color change of the patient's skin 12 at the IV access site 10. Changes in skin color may signify inflammation at the IV access site 10. Thus, if/when the sensors 403 detect skin color changes at the IV access site 10, the system may be configured to communicate with one or both of the infusing equipment and the care provider via, e.g., a communication module 406. The system may then be configured to shut down, e.g., the infusion pump coupled to the tube set 16, send an audible and/or visual alert to the care provider, etc.

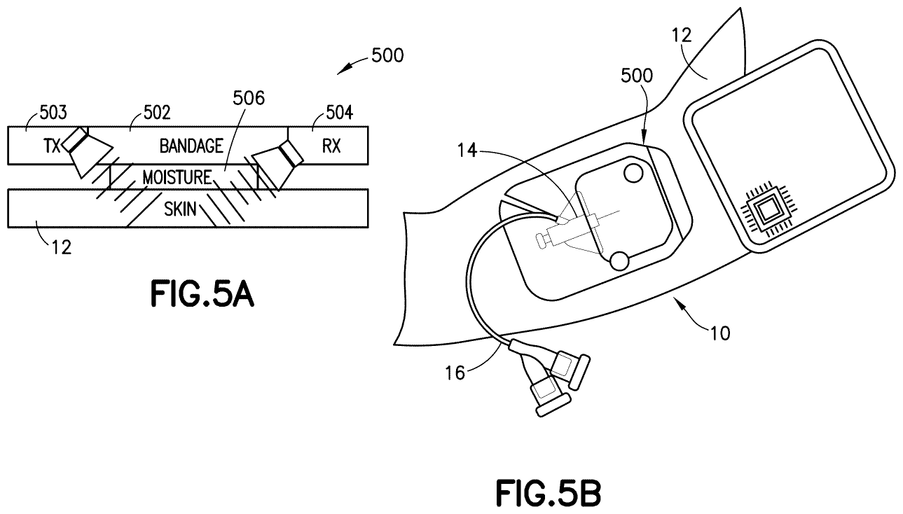

Referring now to FIGS. 5A and 5B, a dressing layer 500 in accordance with another aspect of the present disclosure is shown. Dressing layer 500 includes piezoelectric (PVDF) film transducers embedded in a bandage layer 502, with the PVDF film transducers capable of measuring an acoustic response on the patient's skin 12 so as to detect, e.g., liquid effluent 506.

More specifically, the PVDF film transducers include an acoustic transmitter 503 and an acoustic receiver 504 embedded in the bandage layer 502. Acoustic response on the patient's skin 12 changes based on a number of conditions present at the IV access site 10, including the presence of liquid effluent 506. In this way, the dressing layer 500 detects liquid effluent on the patient's skin at the IV access site 10, the system is configured to communicate with one or both of the infusing equipment and the care provider via, e.g., the wireless communication device (now shown). The system may then be configured to shut down, e.g., the infusion pump coupled to the tube set 16, send an audible and/or visual alert to the care provider, etc., when it is determined that moisture is present below the dressing layer bandage layer 502, signifying a possible complication at the IV access site 10. Accordingly, the dressing layer 500 is configured to continuously monitor the IV access site 10 for at least moisture by way of acoustically-sensitive PVDF film transducers.

In addition to sensing moisture present below the dressing layer 500, the acoustically-sensitive PVDF film transducers may further be capable of sensing swelling of the patient's skin 12 at or near the IV access site 10. That is, the acoustic response of the patient's skin 12 may change with tightening of the skin due to swelling. Thus, if the dressing layer 500 detects swelling of the patient's skin at the IV access site 10 via the acoustic transmitter 503 and the acoustic receiver 504 embedded in the bandage layer 502, the system may be configured to communicate with one or both of the infusing equipment and the care provider via, e.g., the wireless communication device (now shown). The system may then be configured to shut down, e.g., the infusion pump coupled to the tube set 16, send an audible and/or visual alert to the care provider, etc., when it is determined that swelling is present below the bandage layer 502, which signifies a possible complication at the IV access site 10.

Furthermore, in another aspect of the present disclosure, the acoustically-sensitive PVDF film transducers may further be capable of sensing fluid build-up (i.e., infiltration) below the patient's skin 12 at or near the IV access site 10. That is, the acoustic response of the patient's skin 12 may change when a build-up of liquid is present below the skin. Thus, if the dressing layer 500 detects infiltration at or near the IV access site 10 via the acoustic transmitter 503 and the acoustic receiver 504 embedded in the bandage layer 502, the system may be configured to communicate with one or both of the infusing equipment and the care provider via, e.g., the wireless communication device (now shown). The system may then be configured to shut down, e.g., the infusion pump coupled to the tube set 16, send an audible and/or visual alert to the care provider, etc., when it is determined that infiltration is present proximate the IV access site 10.

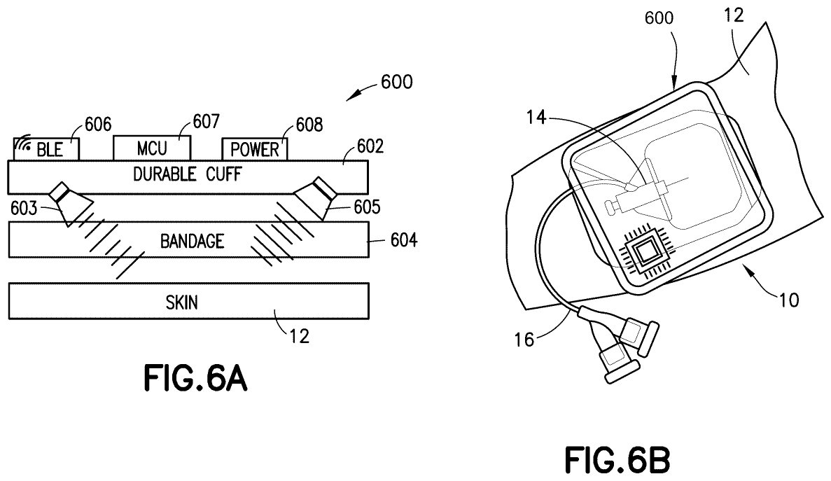

Next, referring to FIGS. 6A and 6B, a cuff and dressing layer assembly 600 in accordance with another aspect of the present disclosure is illustrated. Again, while the dressing layers described above with respect to FIGS. 1A-3B, 5A, and 5B include embedded or otherwise incorporated means of moisture detection, cuff and dressing layer assembly 600 utilizes a separate cuff 602 having ultrasonic transducers 603, 605 embedded therein, with the ultrasonic transducers 603, 605 capable of measuring the acoustic response of, e.g., a dressing layer 604 positioned over an IV access site 10. Accordingly, the dressing layer 604 itself does not need to incorporate sensors, conductive traces, etc., and may be configured as a substantially conventional dressing layer.

As with the other embodiments described above, the cuff and dressing layer assembly 600 is configured to substantially cover the IV access site 10. In some embodiments, the cuff 602 may be configured to extend at least partially around, e.g., the patient's arm in order to remain in place. In addition to embedded ultrasonic transducers 603, 605, the cuff 602 may include various electronics incorporated therein or thereon such as, e.g., a wireless communication device 606 (e.g., a Bluetooth® low energy (BLE) device), a microcontroller 607, a power source 608 (e.g., a battery).

In one embodiment, the cuff and dressing layer assembly 600 may be utilized to detect moisture (i.e., liquid effluent on the patient's skin) surrounding the IV access site 10. That is, the ultrasonic transducers may be configured to both emit and receive acoustic signals, with the acoustic response across the dressing layer 604 and the patient's skin 12 changing in the presence of a liquid effluent. In this way, when cuff 602 detects liquid effluent on the patient's skin at the IV access site 10, the system is configured to communicate with one or both of the infusing equipment and the care provider via, e.g., the wireless communication device 606. The system may then be configured to shut down, e.g., the infusion pump coupled to the tube set 16, send an audible and/or visual alert to the care provider, etc., when it is determined that moisture is present below the dressing layer 604, signifying a possible complication at the IV access site 10. Accordingly, the cuff 602 is configured to continuously monitor the IV access site 10 for moisture, thereby avoiding the need for the care provider to routinely check the IV access site 10 via visual inspection, remove the dressing layer 604, etc.

In addition to sensing moisture present below the dressing layer 600, the cuff 602 having integrated ultrasonic transducers may further be capable of sensing swelling of the patient's skin 12 at or near the IV access site 10. As noted above, the acoustic response of the patient's skin 12 may change with tightening of the skin due to swelling. This tightening of the patient's skin 12 may also cause deformation of the dressing layer 604, which also results in a change of the acoustic response of the dressing layer 604. Thus, if the cuff 602 detects swelling of the patient's skin at the IV access site 10 via the ultrasonic transducers, the system may be configured to communicate with one or both of the infusing equipment and the care provider via, e.g., the wireless communication device (now shown). The system may then be configured to shut down, e.g., the infusion pump coupled to the tube set 16, send an audible and/or visual alert to the care provider, etc., when it is determined that swelling is present below the dressing layer 604, which signifies a possible complication at the IV access site 10.

Furthermore, in another aspect of the present disclosure, the ultrasonic transducers of the cuff 602 may also be capable of sensing fluid build-up (i.e., infiltration) below the patient's skin 12 at or near the IV access site 10. That is, the acoustic response of the patient's skin 12 (and, thus, the dressing layer 604 positioned over the skin) may change when a build-up of liquid is present below the skin. Accordingly, if the cuff 602 detects infiltration at or near the IV access site 10 via the ultrasonic transducers embedded therein, the system may be configured to communicate with one or both of the infusing equipment and the care provider via, e.g., the wireless communication device (now shown). The system may then be configured to shut down, e.g., the infusion pump coupled to the tube set 16, send an audible and/or visual alert to the care provider, etc., when it is determined that infiltration is present proximate the IV access site 10.

Additionally and/or alternatively, the ultrasonic transducers of the cuff 602 may further be utilized to detect degradation (e.g., loss of adhesion, edge lift, etc.) of the dressing layer 604 itself. That is, if the dressing layer 604 begins to degrade and/or deform, resulting in a loss of adhesion, edge lift, etc., the acoustic signal of the dressing is changed. Thus, based on this change in acoustic response, degradation of the dressing layer 604 can be detected. If it is determined that dressing layer degradation has occurred, the system is configured to communicate with one or both of the infusing equipment and the care provider via, e.g., a wireless communication device. The system may then be configured to shut down, e.g., the infusion pump coupled to the tube set 16, send an audible and/or visual alert to the care provider, etc.

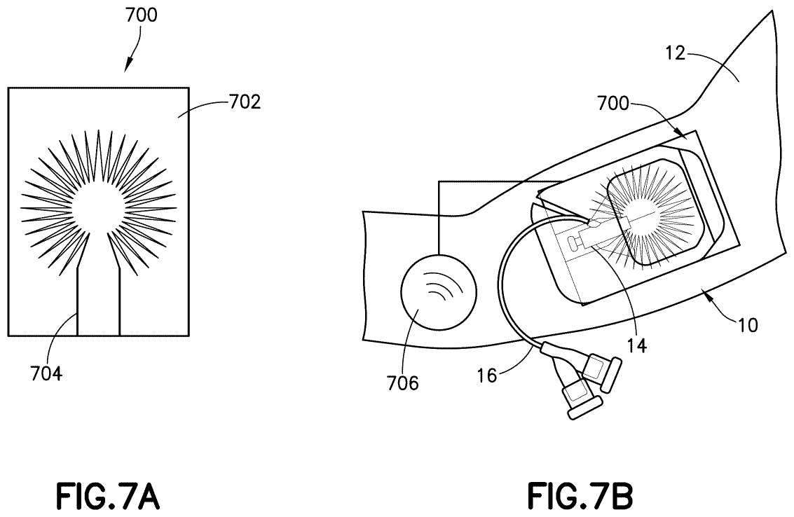

As noted above, it is often desirable to detect swelling of the skin at or around the IV access site, which may signify various potential complications at the IV access site. Accordingly, referring to FIGS. 7A and 7B, a dressing layer 700 in accordance with another aspect of the present disclosure is shown. The dressing layer 700 is configured to substantially cover an IV access site 10 and includes a flexible, primary layer 702 and a plurality of conductive or piezoresistive ink traces 704. The ink traces 704 may be printed on or otherwise embedded in a skin-contact side of the primary layer 702. Furthermore, while not shown, it is to be understood that the ink traces (and the dressing layer 700) are coupled to a power source such as, e.g., a battery or wired connection.

In one aspect of the present disclosure, the ink traces 704 may be utilized to detect swelling in the patient's skin surrounding the IV access site 10. The ink traces 704 undergo a resistance change when in contacting with skin that is swelling, with resistance increasing due to the expansion of the patient's skin 12, which causes movement (and associated strain) in the ink traces 704.

In this way, when the ink traces 704 are used to detect swelling in the patient's skin at the IV access site 10, the system is configured to communicate with one or both of the infusing equipment and the care provider via, e.g., a communication module 706. The system may then be configured to shut down, e.g., the infusion pump coupled to the tube set 16, send an audible and/or visual alert to the care provider, etc., when it is determined that swelling is present below the dressing layer 700, signifying a possible complication at the IV access site 10. Accordingly, the dressing layer 700 is configured to continuously monitor the IV access site 10 for swelling, thereby avoiding the need for the care provider to routinely check the IV access site 10 via visual inspection, remove the dressing layer 700, etc.

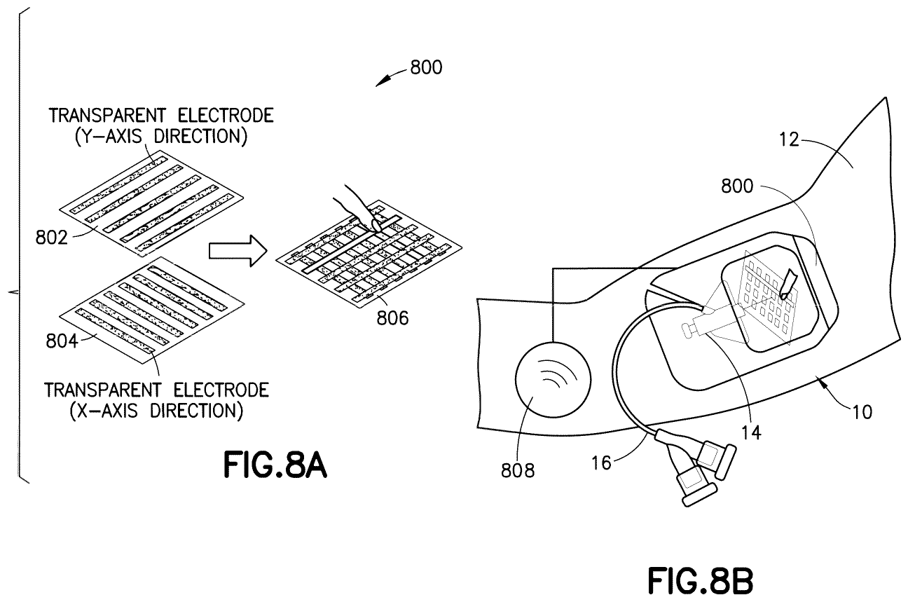

Referring now to FIGS. 8A and 8B, a dressing layer 800 in accordance with another aspect of the present disclosure is shown. Similar to dressing layer 700 described above, dressing layer 800 may be configured to detect swelling at or near the IV access site 10. The dressing layer 800 is configured to substantially cover an IV access site 10 and includes a two flexible layers: a first layer 802 having transparent electrodes extending in a first direction (i.e., a Y-direction), and a second layer 804 having transparent electrodes extending in a second direction (i.e., an X-direction). The first layer 802 and second layer 804 may then be stacked to form a combined layer 806, with combined layer 806 being configured to act as a capacitive touch sensor. While not shown, it is to be understood that the transparent electrodes (and the dressing layer 800) are coupled to a power source such as, e.g., a battery or wired connection.

In one aspect of the present disclosure, the combined layer 806 may be utilized to detect swelling in the patient's skin surrounding the IV access site 10. That is, swelling in the patient's skin causes changes in measured capacitance between the first layer 802 and second layer 804. Thus, when the combined layer 806 is used to detect swelling in the patient's skin at the IV access site 10, the system is configured to communicate with one or both of the infusing equipment and the care provider via, e.g., a communication module 808. The system may then be configured to shut down, e.g., the infusion pump coupled to the tube set 16, send an audible and/or visual alert to the care provider, etc., when it is determined that swelling is present below the dressing layer 800, signifying a possible complication at the IV access site 10. Accordingly, the dressing layer 800 is configured to continuously monitor the IV access site 10 for swelling, thereby avoiding the need for the care provider to routinely check the IV access site 10 via visual inspection, remove the dressing layer 800, etc.

Furthermore, in addition to sensing possible physical complications associated with the IV access site 10, it may also be desirable to sense conditions such as loss of adhesion and/or edge lift of the dressing layer positioned over the IV access site 10. In some embodiments, the combined layer 806 may be utilized to detect deterioration of the dressing layer 800 and its adhesion to the patient's skin, as deterioration of the dressing layer 800 may cause changes in capacitance between the first layer 802 and the second layer 804. Thus, when the combined layer 806 is used to detect deterioration of the dressing layer 800, the system may be configured to communicate with one or both of the infusing equipment and the care provider via, e.g., a communication module 808. The system may then be configured to shut down, e.g., the infusion pump coupled to the tube set 16, send an audible and/or visual alert to the care provider, etc.

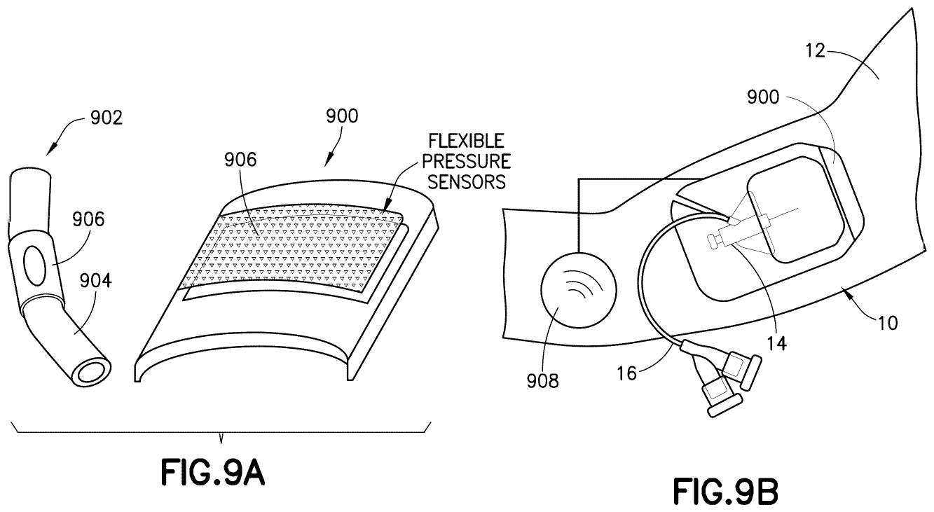

Next, referring to FIGS. 9A and 9B, a dressing layer 900 in accordance with another aspect of the present disclosure is shown. Similar to dressing layers 700 and 800 described above, dressing layer 900 may be configured to detect swelling at or near the IV access site 10. The dressing layer 900 is configured to substantially cover an IV access site 10 and includes one or more sensor members 902, with each sensor member 902 comprising a perforated flexible tube portion 904 and an assembly of piezoresistive film layers 906. The assembly of piezoresistive film layers 906 are configured to change resistance under strain or pressure. While not shown, it is to be understood that the piezoresistive film layers 906 (and the dressing layer 900) may be coupled to a power source such as, e.g., a battery or wired connection.

In one aspect of the present disclosure, the one more sensor members 902 may be utilized to detect swelling in the patient's skin surrounding the IV access site 10. That is, swelling in the patient's skin causes changes in measured resistance in the piezoresistive film layers 906. Thus, when the sensor member(s) 902 are used to detect swelling in the patient's skin at the IV access site 10, the system is configured to communicate with one or both of the infusing equipment and the care provider via, e.g., a communication module 908. The system may then be configured to shut down, e.g., the infusion pump coupled to the tube set 16, send an audible and/or visual alert to the care provider, etc., when it is determined that swelling is present below the dressing layer 900, signifying a possible complication at the IV access site 10. Accordingly, the dressing layer 900 is configured to continuously monitor the IV access site 10 for swelling, thereby avoiding the need for the care provider to routinely check the IV access site 10 via visual inspection, remove the dressing layer 900, etc.

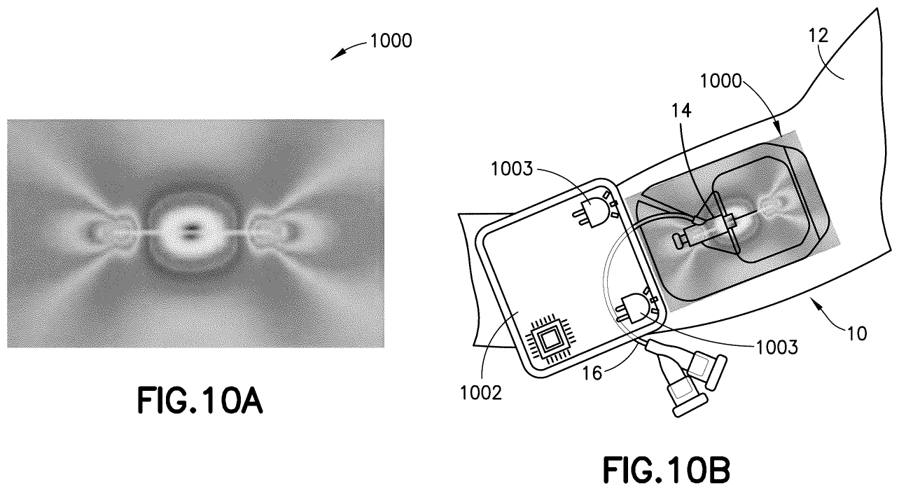

With reference to FIGS. 10A and 10B, a dressing layer 1000 in accordance with another aspect of the present disclosure is shown. The dressing layer 1000 may be configured to detect swelling at or near the IV access site 10. The dressing layer 1000 is configured to substantially cover an IV access site 10 and includes a photoelastic film. The photoelastic film is configured to change optical properties under stress. Thus, swelling in the patient's skin below the dressing layer 1000 may cause changes in the optical properties of the photoelectric film. These changes in optical properties may be determined via photodetectors 1003, which may be positioned on a durable cuff 1002 placed at or near the IV access site 10. While not shown, it is to be understood that the cuff 1002 may include various electronics incorporated therein or thereon such as, e.g., a wireless communication device (e.g., a Bluetooth® low energy (BLE) device), a microcontroller, a power source (e.g., a battery).

Accordingly, when the photoelastic film and photodetectors 1003 are used to detect swelling in the patient's skin at the IV access site 10, the system is configured to communicate with one or both of the infusing equipment and the care provider via, e.g., a wireless communication device. The system may then be configured to shut down, e.g., the infusion pump coupled to the tube set 16, send an audible and/or visual alert to the care provider, etc., when it is determined that swelling is present below the dressing layer 1000, signifying a possible complication at the IV access site 10. Accordingly, the dressing layer 1000 is configured to continuously monitor the IV access site 10 for swelling, thereby avoiding the need for the care provider to routinely check the IV access site 10 via visual inspection, remove the dressing layer 1000, etc.

While the embodiments for smart monitoring of an IV access site described above with respect to FIGS. 1A-10B have each utilized a dressing layer and/or a cuff having monitoring capabilities embedded therein or printed thereon, other embodiments for monitoring the IV access site may include various external monitoring features such as, e.g., an external camera.

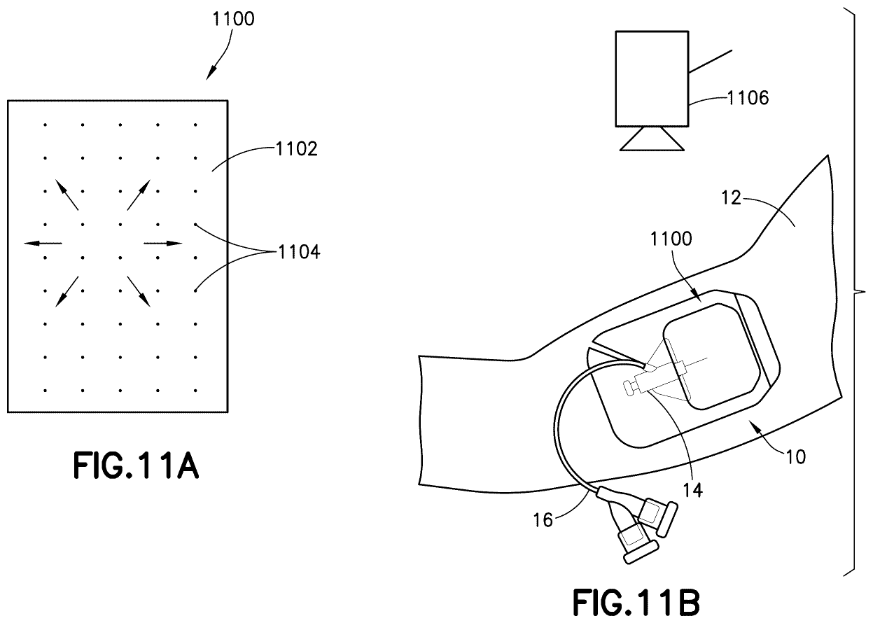

For example, referring to FIGS. 11A and 11B, a dressing layer 1100 incorporating printed marker dots 1104 on a surface 1102 may be utilized to detect, e.g., swelling at the IV access site 10. As shown in FIG. 11A, the printed marker dots 1104 are dispersed at known increments across the surface 1102 of the dressing layer 1100. If and when swelling occurs below the dressing layer 1100, the distance between respective printed marker dots 1104 increases, and such changes in position/distance between printed marker dots 1104 may be continuously measured via a camera 1106 mounted above or beside the dressing layer 1100 and the patient.

Accordingly, based on the measurements between printed marker dots 1104, the camera 1106 is capable of detecting swelling in the patient's skin at the IV access site 10. If it is determined that swelling has occurred, the system is configured to communicate with one or both of the infusing equipment and the care provider via, e.g., a wireless communication device. The system may then be configured to shut down, e.g., the infusion pump coupled to the tube set 16, send an audible and/or visual alert to the care provider, etc., when it is determined that swelling is present below the dressing layer 1100, signifying a possible complication at the IV access site 10. Accordingly, the camera 1106 and dressing layer 1100 are together configured to allow for continuous monitoring of the IV access site 10 for swelling, thereby avoiding the need for the care provider to routinely check the IV access site 10 via visual inspection, remove the dressing layer 1100, etc.

Additionally and/or alternatively, the dressing layer 1100 incorporating printed marker dots 1104 on the surface 1102 may be utilized to detect degradation (e.g., loss of adhesion, edge lift, etc.) of the dressing layer 1100 itself. That is, if the dressing layer 1100 begins to degrade, resulting in a loss of adhesion, edge lift, etc., the distance between respective printed marker dots 1104 changes, and such changes in position/distance between printed marker dots 1104 may be continuously measured via a camera 1106 mounted above or beside the dressing layer 1100 and the patient. Thus, based on the measurements between printed marker dots 1104, the camera 1106 is capable of detecting degradation of the dressing layer 1100. If it is determined that dressing layer degradation has occurred, the system is configured to communicate with one or both of the infusing equipment and the care provider via, e.g., a wireless communication device. The system may then be configured to shut down, e.g., the infusion pump coupled to the tube set 16, send an audible and/or visual alert to the care provider, etc.

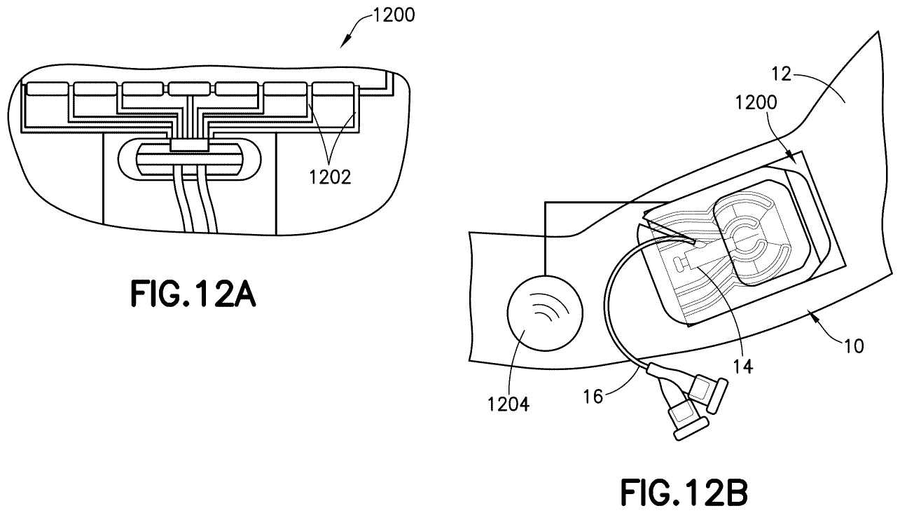

Next, referring to FIGS. 12A and 12B, a dressing layer 1200 in accordance with another aspect of the present disclosure is shown. The dressing layer 1200 includes a plurality of conductive traces 1202 which may be utilized to detect changes in temperature surrounding the IV access site 10. The dressing layer 1200 is configured as a printed thermocouple, with printed silver and carbon forming a thermoelectric junction. Temperature is sensed as a voltage difference between the conductive traces 1202.

In this way, when temperature changes at the IV access site 10, the system is configured to communicate with one or both of the infusing equipment and the care provider via, e.g., a communication module 1204. The system may then be configured to shut down, e.g., the infusion pump coupled to the tube set 16, send an audible and/or visual alert to the care provider, etc., when it is determined that there is a predetermined change in temperature below the dressing layer 1200, signifying a possible complication (e.g., an infection) at the IV access site 10.

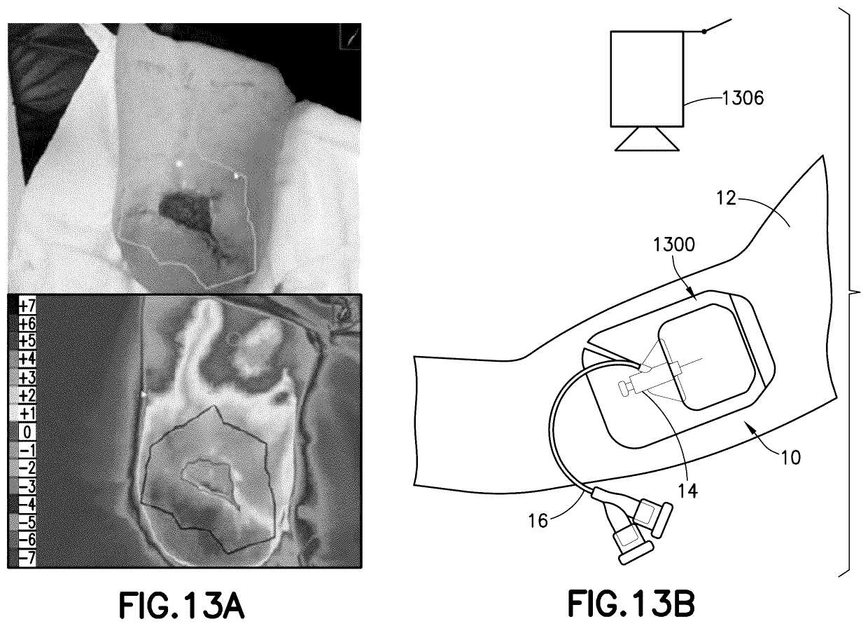

Referring now to FIGS. 13A and 13B, a system utilizing machine vision IR temperature sensing is illustrated. The system includes a dressing layer 1300 positioned at the IV access site 10, as well as an external IR camera 1306 positioned on, e.g., a gantry or cart at a position above or beside the IV access site 10. The IR camera 1306 may be a high-resolution IR camera, and may be coupled to an external processor (not shown) having software capable of producing images illustrating temperature differentials at a target site (such as that which is shown in FIG. 13A).

Accordingly, based on the temperature differentials determined by the IR camera 1306 and associated processor, an undesirable variation and/or rise in temperature may be detected at the IV access site 10. In such a scenario, the system may be configured to communicate with one or both of the infusing equipment and the care provider via, e.g., a wireless communication device. The system may then be configured to shut down, e.g., the infusion pump coupled to the tube set 16, send an audible and/or visual alert to the care provider, etc., when it is determined that an undesirable temperature differential is present below the dressing layer 1300, signifying a possible complication at the IV access site 10. Accordingly, the IR camera 1306 allows for continuous monitoring of the IV access site 10 for temperature variations, thereby avoiding the need for the care provider to routinely check the IV access site 10 via visual inspection, remove the dressing layer 1300, etc.

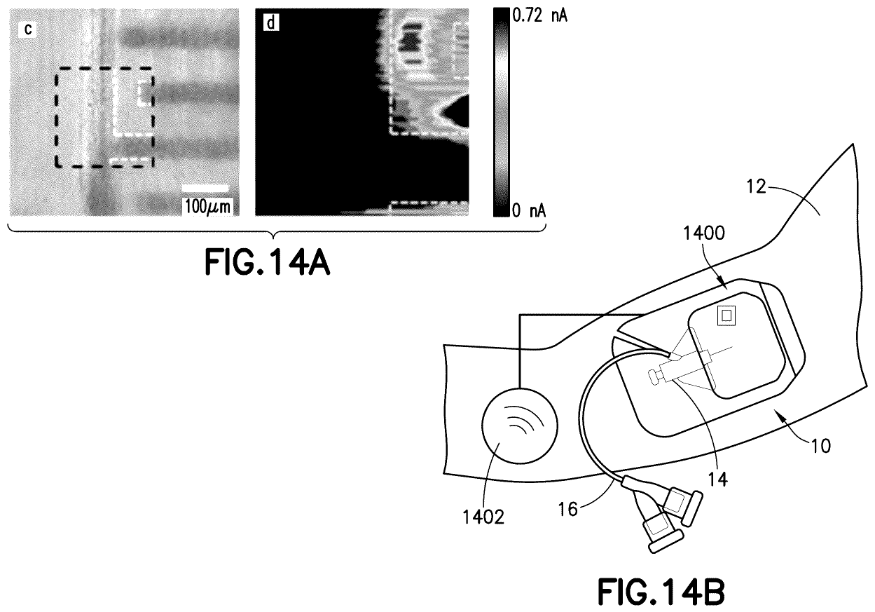

Next, referring to FIGS. 14A and 14B, a dressing layer 1400 in accordance with another aspect of the present disclosure is shown. The dressing layer 1400 includes a plurality of printed photodetectors (as shown in FIG. 14A), which may be utilized to detect changes in temperature surrounding the IV access site 10. In particular, the dressing layer 1400 may incorporate an array of ˜1 mm graphene and MoS2 printed photodetectors, with temperature differentials being sensed by the photodetectors.

In this way, when temperature differentials above a certain threshold are determined at the IV access site 10, the system is configured to communicate with one or both of the infusing equipment and the care provider via, e.g., a communication module 1402. The system may then be configured to shut down, e.g., the infusion pump coupled to the tube set 16, send an audible and/or visual alert to the care provider, etc., when it is determined that there is a predetermined change and/or differential in temperature below the dressing layer 1400, signifying a possible complication (e.g., an infection) at the IV access site 10.

Additionally and/or alternatively, the plurality of printed photodetectors may be utilized to detect changes in skin color surrounding the IV access site 10. As noted above, changes in skin color may signify inflammation at the IV access site 10. Thus, if/when the printed photodetectors of dressing layer 1400 detect skin color changes at the IV access site 10, the system may be configured to communicate with one or both of the infusing equipment and the care provider via, e.g., a communication module 1402. The system may then be configured to shut down, e.g., the infusion pump coupled to the tube set 16, send an audible and/or visual alert to the care provider, etc.

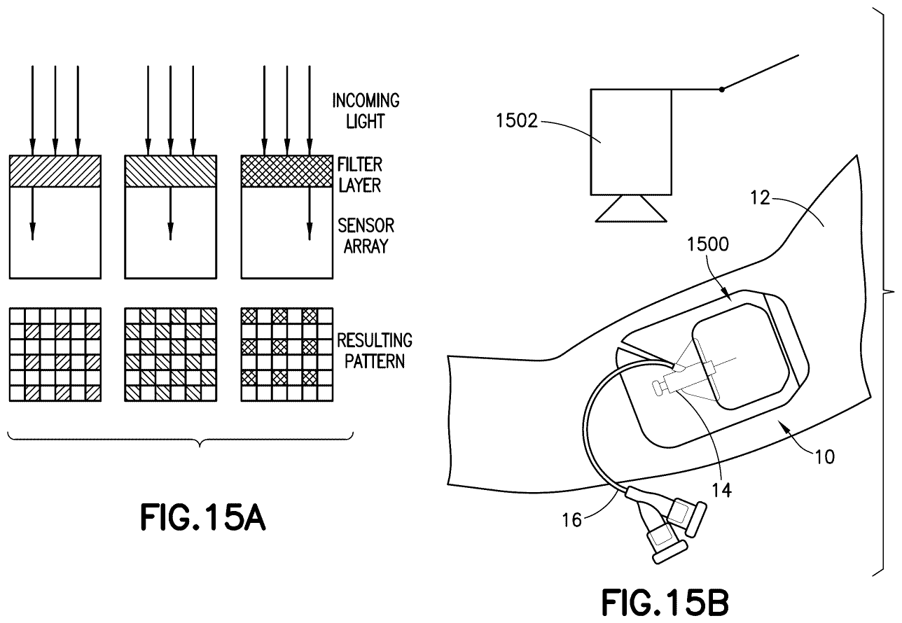

Referring to FIGS. 15A and 15B, a system utilizing machine vision color sensing is illustrated. The system includes a dressing layer 1500 positioned at the IV access site 10, as well as an external high-resolution camera 1502 positioned on, e.g., a gantry or cart at a position above or beside the IV access site 10. The camera 1502 may include various color filters (as shown in FIG. 15A) and may be coupled to an external processor (not shown) having software capable of producing images identifying color differentials at a target site such as, e.g., the IV access site 10.

Accordingly, based on the color differentials determined by the camera 1502 and associated processor, an undesirable variation in color at or near the IV access site 10 may be detected. In such a scenario, the system may be configured to communicate with one or both of the infusing equipment and the care provider via, e.g., a wireless communication device. The system may then be configured to shut down, e.g., the infusion pump coupled to the tube set 16, send an audible and/or visual alert to the care provider, etc.

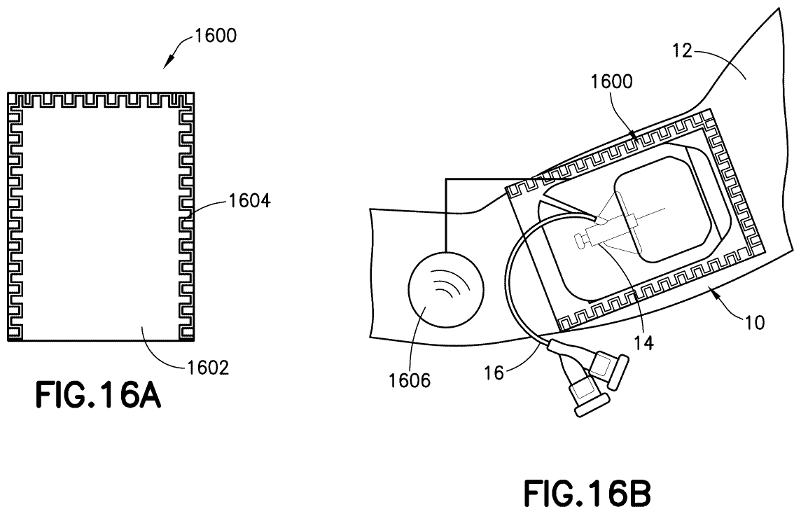

In addition to sensing possible physical complications associated with the IV access site 10, it may also be desirable to sense conditions such as loss of adhesion and/or edge lift of the dressing layer over the IV access site 10 and/or movement or displacement of the catheter. Accordingly, referring to FIGS. 16A and 16B, a dressing layer 1600 in accordance with another aspect of the present disclosure is illustrated. Dressing layer 1600 is configured to substantially cover an IV access site 10 and includes a flexible, primary layer 1602 and a conductive trace perimeter 1604. The conductive trace perimeter 1604 may be printed on or otherwise embedded in either side of the primary layer 1602. Furthermore, while not shown, it is to be understood that the conductive trace perimeter 1604 (and the dressing layer 1600) are coupled to a power source such as, e.g., a battery or wired connection.

In one aspect of the present disclosure, the conductive trace perimeter 1604 may be utilized to detect degradation and/or loss of adhesion of the dressing layer 1600. That is, the conductive trace perimeter 1604 undergoes an increase in resistance when the edge of the dressing layer 1600 degrades (i.e., loss of adhesion to the patient's skin, lifting of the edges, etc.). In this way, when conductive trace perimeter 1604 detects such a degradation based on this increase in resistance, the system is configured to communicate with one or both of the infusing equipment and the care provider via, e.g., a communication module 1606. The system may then be configured to shut down, e.g., the infusion pump coupled to the tube set 16, send an audible and/or visual alert to the care provider, etc.

Similarly, the conductive trace perimeter 1604 of dressing layer 1600 may be utilized to detect movement/displacement of the catheter in place at the IV access site 10. The conductive trace perimeter 1604 surrounding the catheter may be degraded by catheter movement, thereby increasing the resistance detected by the conductive trace perimeter 1604. Thus, when conductive trace perimeter 1604 detects such a degradation based on catheter movement, the system is configured to communicate with one or both of the infusing equipment and the care provider via, e.g., a communication module 1606. The system may then be configured to shut down, e.g., the infusion pump coupled to the tube set 16, send an audible and/or visual alert to the care provider, etc.

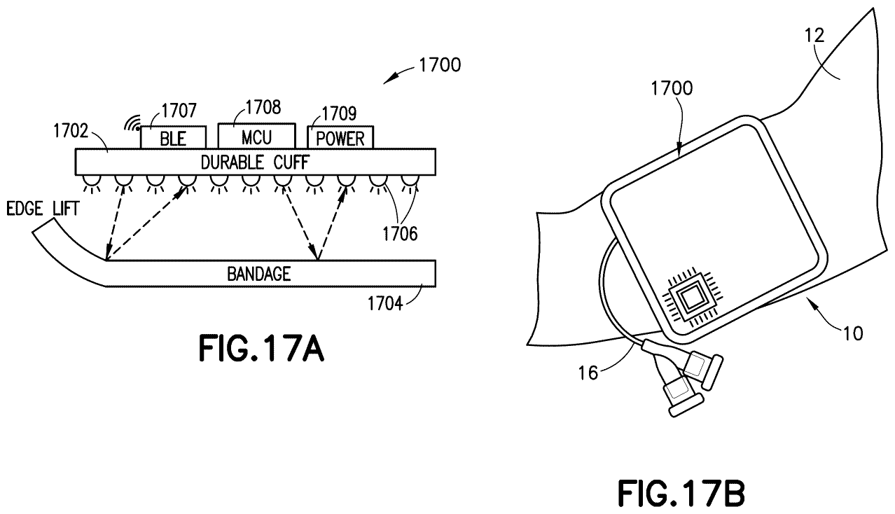

Referring to FIGS. 17A and 17B, a cuff and dressing layer assembly 1700 in accordance with another aspect of the present disclosure is illustrated. The cuff and dressing layer assembly 1700 utilizes a cuff 1702 having a plurality of optical sensors 1706 embedded thereon. Accordingly, the dressing layer 1704 itself does not need to incorporate sensors, conductive traces, etc., and may be configured as a substantially conventional dressing layer.

As with the other embodiments described above, the cuff and dressing layer assembly 1700 is configured to substantially cover the IV access site 10, which is the site on the patient's skin 12 in which the IV catheter (not shown) is inserted. In some embodiments, the cuff 1702 may be configured to extend at least partially around, e.g., the patient's arm in order to remain in place. In addition to embedded optical sensors 1706, the cuff 1702 may include various electronics incorporated therein or thereon such as, e.g., a wireless communication device 1707 (e.g., a Bluetooth® low energy (BLE) device), a microcontroller 1708, a power source 1709 (e.g., a battery).

In one aspect of the present disclosure, the cuff and dressing layer assembly 1700 may be utilized to detect degradation of the dressing layer 1704 via, e.g., loss of adhesion, edge lift, etc. That is, the optical sensors 1706 may be configured to both emit and detect light, with the reflectance of light from the dressing layer 1704 changing when the dressing is deformed. In this way, when cuff 1702 detects deformation of the dressing layer 1704, the system is configured to communicate with one or both of the infusing equipment and the care provider via, e.g., the wireless communication device 1707. The system may then be configured to shut down, e.g., the infusion pump coupled to the tube set 16, send an audible and/or visual alert to the care provider, etc.

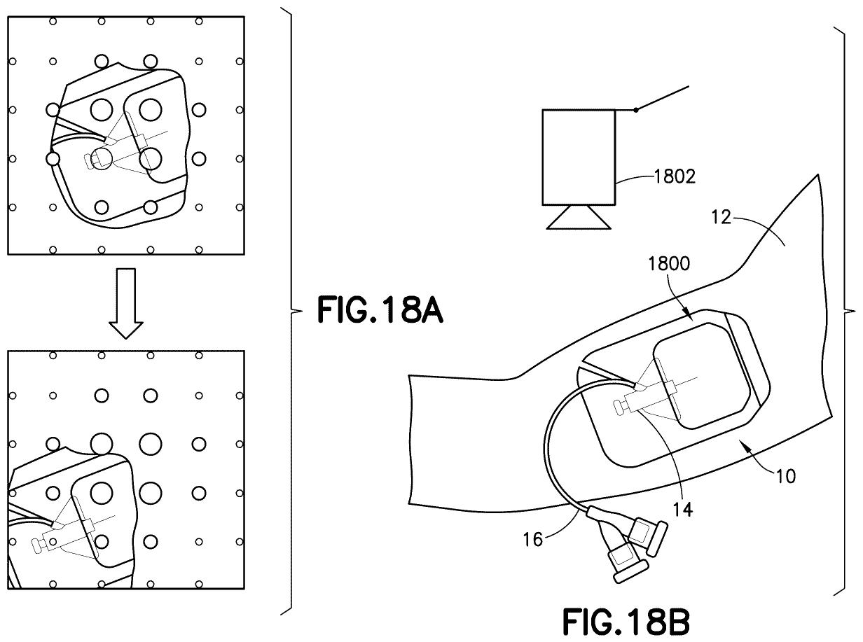

Referring now to FIGS. 18A and 18B, a system utilizing machine vision catheter movement tracking is illustrated. The system includes a dressing layer 1800 positioned at the IV access site 10, as well as an external high-resolution camera 1802 positioned on, e.g., a gantry or cart at a position above or beside the IV access site 10. The camera 1802 may be coupled to an external processor (not shown) having software capable of producing images identifying movement of the catheter relative to a known position (as shown in FIG. 15A).

Accordingly, based on the images captured by the camera 1802, undesirable movement of the catheter relative to the IV access site 10 may be detected. In such a scenario, the system may be configured to communicate with one or both of the infusing equipment and the care provider via, e.g., a wireless communication device. The system may then be configured to shut down, e.g., the infusion pump coupled to the tube set 16, send an audible and/or visual alert to the care provider, etc.

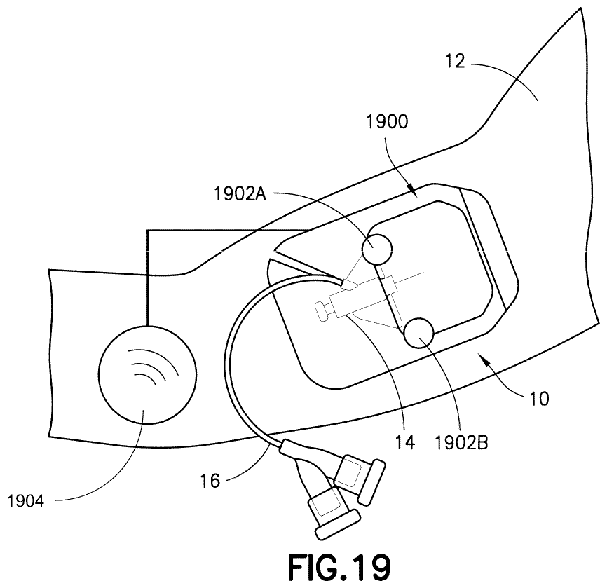

Next, referring to FIG. 19, a dressing layer 1900 in accordance with another aspect of the present disclosure is illustrated. The dressing layer 1900 includes at least two electrodes 1902A, 1902B, with the catheter 14 acting as a dielectric medium between the electrodes 1902A, 1902B. In this way, the electrodes 1902A, 1902B may be utilized to detect movement/displacement of the catheter 14 in place at the IV access site 10, as movement of the catheter 14 changes the capacitance between the electrodes 1902A, 1902B. Thus, when a predetermined change in capacitance between the electrodes 1902A, 1902B is detected, it is assumed that such a change is based on unwanted catheter movement/displacement.

Accordingly, the system is configured to communicate with one or both of the infusing equipment and the care provider via, e.g., a communication module 1904. The system may then be configured to shut down, e.g., the infusion pump coupled to the tube set 16, send an audible and/or visual alert to the care provider, etc.

While several embodiments of smart monitoring of an IV access site were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A system for monitoring an intravenous (IV) access site for catheter insertion into a patient's skin, the system comprising:
a dressing layer configured to cover at least a portion of the IV access site,
wherein the dressing layer comprises at least one embedded component configured for use in detecting one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer.

2. The system of claim 1, wherein the at least one embedded component comprises a plurality of conductive traces.

3. The system of claim 1, wherein the at least one embedded component comprises a capacitive film layer.

4. The system of claim 1, wherein the at least one embedded component comprises at least two piezoelectric film transducers.

5. The system of claim 1, wherein the at least one embedded component comprises a plurality of conductive or piezoresistive ink traces.

6. The system of claim 1, wherein the at least one embedded component comprises a first layer having transparent electrodes extending in a first direction and a second layer having transparent electrodes extending in a second direction perpendicular to the first direction.

7. The system of claim 1, wherein the at least one embedded component comprises a plurality of piezoresistive film layers.

8. The system of claim 1, wherein the at least one embedded component comprises a photoelastic film.

9. The system of claim 1, wherein the at least one embedded component comprises an array of printed photodetectors.

10. The system of claim 1, wherein the at least one embedded component comprises at least two electrodes configured to be positioned on opposite sides of the catheter.

11. The system of claim 1, wherein the dressing layer further comprises an integrated power source and communication electronics.

12. The system of claim 1, wherein, based upon the detection of one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer, the system is further configured to initiate at least one of the shutdown of an infusion pump fluidly coupled the catheter and send at least one of an audible or visual alert to a care provider.

13. A system for monitoring an intravenous (IV) access site for catheter insertion into a patient's skin, the system comprising:
a dressing layer configured to cover at least a portion of the IV access site; and
a cuff configured to be disposed at least partially around the dressing layer,
wherein the cuff comprises at least one embedded component configured for use in detecting one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer.

14. The system of claim 13, wherein the at least one embedded component comprises a plurality of optical sensors.

15. The system of claim 13, wherein the at least one embedded component comprises a pair of ultrasonic transducers.

16. The system of claim 13, wherein the at least one embedded component comprises at least two photodetectors.

17. The system of claim 13, wherein the cuff further comprises a wireless communication device, a microcontroller, and a power source.

18. The system of claim 13, wherein, based upon the detection of one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer, the system is further configured to initiate at least one of the shutdown of an infusion pump fluidly coupled the catheter and send at least one of an audible or visual alert to a care provider.

19. A system for monitoring an intravenous (IV) access site for catheter insertion into a patient's skin, the system comprising:
a dressing layer configured to cover at least a portion of the IV access site; and
a camera positioned externally from the IV access site and configured to capture images of the IV access site,
wherein the camera is configured for use in detecting one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer.

20. The system of claim 19, wherein the camera is a high-resolution photographic camera.

21. The system of claim 19, wherein the camera is an infrared camera.

22. The system of claim 19, wherein, based upon the detection of one or more of swelling of the IV access site, temperature changes at the IV access site, fluid infiltration/extravasation from the catheter, fluid detection on top of the patient's skin at a skin piercing location, catheter dislodgement, color changes of the patient's skin at the IV access site, presence of microbes at the IV access site, and degradation of the dressing layer, the system is further configured to initiate at least one of the shutdown of an infusion pump fluidly coupled the catheter and send at least one of an audible or visual alert to a care provider.
